(19) United States
(12) Patent Application Publication
(10) Pub. No.: US 2010/0256649 A1
(43) Pub. Date: Oct. 7, 2010

(54) METHOD FOR PRODUCING A DRILL ASSISTANCE DEVICE FOR AT LEAST ONE IMPLANT HOLE IN A BONE STRUCTURE AND DEVICE OBTAINED

(76) Inventors: Jean Capsal, Lacougotte-Cadoul (FR); Alain Fernandez Scoma, Montastruc La Conseillere (FR)

Correspondence Address: CHRISTIE, PARKER & HALE, LLP, PO BOX 7068, PASADENA, CA, 91109-7068, US

(21) Appl. No.: 12/602,634

(22) PCT Filed: Jun. 3, 2008
(86) PCT No.: PCT/IB2008/052161

§ 371 Date: Jun. 11, 2010

Related U.S. Application Data
(60) Provisional application No. 60/982,152, filed on Oct. 24, 2007.

(30) Foreign Application Priority Data
Jun. 4, 2007 (FR) 0703939

(51) Int. Cl. A61B 17/56; B23P 17/04

(52) U.S. Cl. 606/96; 29/592

(57) ABSTRACT

According to this method, one makes a three-dimensional solid reproduction (12) of the anatomical zone of implantation and, from this reproduction, a drill assistance device (30) comprising a rigid base (35) provided with at least one drill guide (34, 38). According to the invention,—the three-dimensional solid reproduction (12) consists of a reproduction of an internal and peripheral portion of said bone structure, exempt, at least in the vicinity of each implant hole, from the surrounding non-osseous tissues and mucosa,—said rigid base (35) having at least one surface, called contact surface, adapted to be able to bear in contact with an external surface portion of the three-dimensional solid reproduction (12), and therefore also later in contact with a peripheral surface portion of said bone structure.

The invention concerns a method for producing a device, called drill assistance device, for assisting with the positioning and production by drilling of at least one implant hole in a bone structure of a patient for the placement of at least one implant. It extends to such an assistance device for drilling implant holes.

The realization of drillings of bone implant holes—in particular but not exclusively for the placement of dental implants—is a particularly delicate operation. Indeed, each drilling must be done with very great precision with regard to the placement of the drilling on the surface of the bone structure, the axial orientation of the drilling and the depth of the drilling. These different parameters are first crucial with regard to the later functionality of the implant, in particular from a mechanical perspective. Thus, the implant holes must be made in portions of the bone structure which are as solid and clean as possible. However, traditionally, the placement of an implant takes place after other attempts at therapeutic treatments, and therefore on a physiological site which is often damaged.

Moreover, it is essential for the realization of the drilling not to lead to lesions, and in particular not to interfere with the nervous, ligamentary, muscular or other parts extending near or on the periphery of the bone structure. However, achieving this objective remains particularly delicate on certain implant sites, in particular in the field of dental implants in which it is necessary in particular to avoid any attack on the dental nerve or the sinuses.

It is also necessary to ensure maximum preservation of the integrity of the bone structure when the drilling is performed, and in particular by avoiding any overheating phenomenon and ensuring proper evacuation of drill dust.

Until now, these objectives have only been achieved in practice through the knowledge and skill of the practitioner.

Of course, many propositions have been made aiming to exploit modern imaging and computer technologies to make this type of intervention reliable and guarantee it.

A first considered solution (cf. U.S. Pat. No. 5,320,529) consists of proposing a drill guide made of one piece placed in the patient's mouth and having drill guide bores. This drill guide can be made from a stereolithographic model previously manufactured from tomographic data and reproducing the implant site, this model being supposed to allow the surgeon to optimize the placement of the implant holes. However, verification of this placement is done through occlusive radiography, which does not take into account the superposition of planes or deformations caused by the imprecision of the radiographic axes. And contrary to the hypothesis taken in this document, the dental nerve is not laterally offset but is unfortunately central, and in the desired drill axis for implants, which does not truly allow verification of the placement by radiography.

Moreover, this drill guide is placed in the patient's mouth on the gum and is not precisely positioned relative to the bone structure, nor is it rigidly fixed to the latter when the first drilling is performed. Indeed, in the solution proposed in this document, the drill guide is supposed to be able to be fixed initially through placement of the first implant. Moreover, this guide does not allow, once the first implant is placed, irrigation—in particular external—of the osseous tissues during drilling, or evacuation of dust. Yet it has been shown that the presence of good irrigation during bone drilling is essential to avoid overheating of the bone, primary cause of later non-osseo-integration of the implant. Moreover, residual drill dust can cause difficulties in placing the implant. As a result, the uncertainties in the production and use of such a drill guide proposed since 1992 are such that it has not been able to be the object of practical exploitation to date.

Given the presumed performance of current computer technologies, a very large number of solutions have been proposed which tend, on the contrary, to exploit virtual imaging to design the shape and the dimensions of implants, and/or to produce drill guides directly from tomographic data. Moreover, a large number of solutions have also been proposed which use drill guides allowing surgery without flaps, i.e. with drill guides fixed to the neighboring dental structure or to the gum. As an illustration of such solutions, we can cite, for example, WO 2007/015140; EP 0756735; WO 03/071972; EP 1364625; U.S. Pat. No. 6,704,439; WO 2005/0558556 and the Internet sites:

www.nobelbiocare.com/global/fr/ClinicalProcedures/NobelGuide/default.htm; www.fcldentaire.com/DossImplanto/guide.htm; . . .

It is important to note, to this end, that the English expressions “jawbone” or “jaw bone structure”, as they are used in particular in all of these documents, always designate the entire maxilla, including the gum, and not only the bone structure of the jawbone.

However, the inventors have determined, counter to all of these generalized current recommendations, that all solutions which bear on the gum necessarily involve an unacceptable imprecision during surgical implementation and must necessarily be banned. Moreover, these guides do not allow visual monitoring of the penetration of the drill into the bone structure, and do not allow irrigation during drilling, or evacuation of drill dust.

Moreover, the inventors have demonstrated that tomographic data not being able to represent a real surgical site with perfect accuracy, it is in reality illusory to seek to define the shape of the dimensions of implants or implant guides directly from digital imaging data. In fact, with these techniques, despite the use of a great computer precision, it has been reported that, in practice, up to 30% of implants can perforate the maxillary sinus or the inferior dental nerve.

Furthermore, the internet document www.materialise.com/materialise/download/en/490061/file pp 55-60 describes a guide variation designed entirely from imaging and able to be directly supported on the bone structure. Nevertheless, for the same reasons as stated above, the inventors have determined that the precision which can be obtained in the production of such a guide is not sufficient. Moreover, it is not possible to take the presence of concave areas or undercut surface portions (hindering or preventing the placement of the guide) into account, or to correctly assess the quality of the bone structure, using only data from tomographic imaging.

Furthermore, a guide of this type does not in reality allow irrigation—in particular external—of the part of the bone covered by the guide during drilling and placement of the implant. Because of this, the abovementioned heating problem arises in the same way, and is even amplified by the fact that the bone exposed without irrigation is subject to drying able to cause necrotic-type complications. For the evacuation of dust and irrigation, this known technique recommends periodically removing the guide during the operation, which makes it impossible to guarantee a precise replacement of the guide, and in practices leads to obtaining imperfect implant holes. Indeed, a repositioning imprecision invisible to the naked eye or by the practitioner can lead to flaws in the diameter of the drilling of values sufficiently large to prevent primary anchoring of the threads of the implant. This problem arises even more acutely in the case of conical implants.

Under these conditions, the invention aims to propose a drill assistance device and a method for producing same, with which any positioning and drilling errors are impossible with regard to the positioning, the orientation and the depth of drilling, and which make it possible to acquire a permanent visual monitoring by the practitioner, and perfect external and internal irrigation of the drilling.

The invention also aims to propose one such device, and method for producing same, which make it possible to increase the speed with which the surgical implant operation is performed considerably while also ensuring its complete safety, while protecting the patient from any wrong manipulation by the practitioner.

To do this, the invention concerns a method for producing a device, called a drill assistance device, for assisting with the positioning and production by drilling of at least one implant hole in a bone structure of a patient for the placement of at least one implant, in which:
one makes, from tomographic data representative of an anatomical zone including said bone structure, a three-dimensional solid reproduction of said anatomical zone;
one makes, from the three-dimensional solid reproduction, a drill assistance device comprising a rigid base able to position itself and fix itself on the three-dimensional solid reproduction, this drill assistance device comprising, for each drilling to be done, at least one drill guide able to define the position, the axial orientation and the depth of this drilling, each drill guide being rigidly supported by the base,

characterized in that
the three-dimensional solid reproduction consists of a reproduction of an internal and peripheral portion of said bone structure, exempt, at least in the vicinity of each implant hole, from the surrounding non-osseous tissues and mucosa,
the three-dimensional solid reproduction is made with sufficient precision to reproduce the surface irregularities and the internal density variations of the bone structure,
said rigid base having at least one surface, called contact surface, adapted to be able to bear in contact with an external surface portion of the three-dimensional solid reproduction, and therefore also later in contact with a peripheral surface portion of said bone structure,

said contact surface having a surface state adapted to fit the surface irregularities of this surface portion and so as to be able to fit onto this surface portion while being rigidly maintained in relation to said three-dimensional solid reproduction, and therefore also later on said bone structure, at least in part thanks to this fitting, in one single possible location.

The invention also extends to a device, called drill assistance device, for assisting with the positioning and the production by drilling of at least one implant hole in a bone structure of a patient for the placement of at least one implant, comprising:
for each drilling to be done, at least one drill guide able to define the position, the axial orientation and the depth of this drilling,
a rigid base having at least one surface, called contact surface, adapted to bear in contact with a peripheral surface portion of said bone structure, each drill guide being rigidly supported by the base,

characterized in that said contact surface of the rigid base has a surface state adapted to fit the surface irregularities of said peripheral surface portion of the bone structure and so as to be able to fit onto this surface portion while being rigidly maintained relative to said bone structure at least in part thanks to this fitting, in one single possible location.

The invention thus rests on the combination on one hand of a drill assistance device of the type coming directly into contact with the surface of the bone structure, on the other hand on the fact of providing a base for this device having a contact surface with the bone structure which is sufficiently precise with regard to its surface state for this base to be able to closely fit the surface state of the bone structure and fit in one single possible location, and on the fact of producing this drill assistance device not directly from tomographic data or digital imaging data, but from a three-dimensional solid reproduction of the bone structure itself, the latter being produced with sufficient precision. Thus, the invention goes doubly counter to the prior state of the art, according to which on one hand one should avoid, at all costs, the surgical act of expulsion of the bone surface, and on the other hand one tries to produce the drill guide directly from digital imaging data.

Thanks to this, the invention makes it possible for the first time to perform in practice, with great speed of execution, with no risk of error, placement of implants—in particular dental implants—in extremely damaged and/or poorly accessible and/or cramped (having small dimensions) sites.

Advantageously and according to the invention, said three-dimensional solid reproduction is a stereolithographic reproduction—i.e. obtained through a method for rapid production of prototypes. Preferably, said three-dimensional solid reproduction is made in a translucent or transparent material making it possible to visualize the penetration of drills into this material.

Moreover, advantageously and according to the invention, one produces said three-dimensional solid reproduction such that it has all of the areas of the bone structure liable to receive an implant hole.

Advantageously and according to the invention, said three-dimensional solid reproduction is produced with a manufacturing uncertainty relative to the actual dimensions of the bone structure portion reproduced less than or equal to 500 μm—in particular in the vicinity of 400 μm. To do this, in particular, in a method according to the invention, one uses tomographic data representing slices of said anatomical area with a distance between the slices less than or equal to 500 μm—in particular in the vicinity of 400 μm.

Advantageously, in a method according to the invention, after having produced said three-dimensional solid reproduction, one produces a model of the drill assistance device at least in part through modeling on the three-dimensional solid reproduction, this model being adapted to be able to then be reproduced by molding to produce the drill assistance device. At least, one model of the base is modeled on the three-dimensional solid reproduction. To do this, one makes, for each implant hole, a drill hole in the three-dimensional solid reproduction, in one position, along an axis and with a depth corresponding to the position, the axis and the depth of the implant hole, respectively, then one places, in each drill hole, a positioning rod, then one produces a model of the drill assistance device by placing a model of each drill guide around each positioning rod and modeling a base model around each drill guide model thus placed, then one produces the drill assistance device through molding from this model.

In one advantageous embodiment according to the invention, each drill guide comprises a tubular bush rigidly extending axially to extend the base while defining the position and the axial orientation of the drilling. A tubular bush of this type may be formed in a single piece with the base, i.e. made up of an extension of the base. Moreover, each bush is adapted to be able to receive a tubular rigid sleeve for guiding the drill, this rigid sleeve being able to be inserted axially into the bush until it abuts in a position where it is maintained rigidly without play relative to the bush, each guide sleeve having a cylindrical internal bore with a diameter adapted to be able to receive and axially guide, without play, a drill, the axial length of each guide sleeve being adapted to have a free stop end in contact with which a drill tool may come at the end of drilling, so as to prohibit any excess drilling in depth.

Moreover, advantageously and according to the invention, the base has at least one opening adapted to allow the visualization of the penetration of a drill into the bone structure, the evacuation of drill dust and the passage of an irrigation fluid. Advantageously, such an opening is arranged across from the base of a drill guide to allow visualization of a drill introduced into this bush or into a guide sleeve supported by this bush, the evacuation of waste and the external irrigation during drilling with a drill in this bush.

Advantageously and according to the invention, each drill guide has a free stop end in contact with which a drill tool may come at the end of drilling, the axial length of the drill guide being adapted to prevent any excess drilling in depth.

Advantageously and according to the invention, each drill guide—in particular each bush and each guide sleeve, has, from its free stop end, at least one axial portion in the shape of a half-cylinder of revolution adapted to allow the lateral insertion of a drill into this drill guide. The bush can, in some variations of embodiment, be made up of only one cylinder of revolution portion, the lateral opening of the bush extending in the extension of an opening arranged in the base.

Advantageously and according to the invention, the base has at least one perforation for the passage of a fixing member (screw or rivet) into the bone structure. Thus, the drill assistance device according to the invention can be rigidly fixed on the bone structure by one or several screw(s) and/or one or several rivet(s).

Advantageously and according to the invention, one produces the base such that it bears on the cortical parts of the bone structure allowing the assembly and disassembly of the base and across from the areas of maximum bone density.

The drill assistance device according to the invention can be made of any suitable dietary sterilizable rigid material. Preferably, this is a metallic material such as an alloy chosen among rigid polymers (polycarbonates, polymethacrylates, fluorocarbon polymers such as PTFEs), and metal alloys chosen among chrome, cobalt, nickel, copper, platinum, titanium, gold.

The invention is applicable in particular to the production of a drill assistance device adapted to allow the production of at least one dental implant hole, the bone structure being a maxillary structure.

A drill assistance device according to the invention is also characterized by all or part of the characteristics mentioned above in reference to the method according to the invention.

In particular, advantageously and according to the invention, the surface state of said contact surface of the rigid base reproduces surface irregularities with a precision less than or equal to 500 μm—in particular in the vicinity of 400 μm.

According to another characteristic of the invention, the base has a reduced thickness, in the vicinity of 1 to 2 mm, relative to the height of each drill guide; this height of each drill guide is in the vicinity of 5 to 15 mm.

According to another characteristic of the invention, at least one aforementioned opening is connected to the base of at least one drill guide, with which it communicates. In this case, said opening can be arranged so as to extend over at least one third of the area for connecting the drill guide to the base.

Such an opening can also be remote from the base of at least one drill guide, and not communicate with this base.

According to another characteristic of the invention, at least one aforementioned opening has a length and a width, this length and this width being between three times and ten times the internal diameter of the drill guide across from the base of which this opening is arranged. This opening thus has dimensions making it possible to truly and perfectly visualize the penetration area of a drill in the bone structure and/or the reference marks present on this drill, and enabling a perfect introduction and evacuation of the irrigation fluid as well as a perfect evacuation of drill dust.

According to another characteristic of the invention, the dimensions of at least one opening are such that the opening defines, with the edges of the base, a peripheral border strip.

According to another characteristic of the invention, at least one drill guide is formed of a cylinder of revolution portion, defining a lateral opening, and an opening arranged in the base extends in an extension of this lateral opening.

According to another characteristic of the invention, at least one drill guide is made up solely of one cylinder of revolution portion. In this case, the drill guide can comprise said bush and this bush may be made up only of said cylinder of revolution portion and comprise an annular stop completing the contour of this cylinder of revolution portion. Said annular stop can extend away from the area for connecting the bush to the base, in particular around one third of the height of this bush. Said annular stop can be made up of an annulus portion. Said annular stop can also be made up of a radial surface of an axial end of one portion of the base of the bush, extending from the base.

According to another characteristic of the invention, the drill guide comprises the aforementioned guide sleeve, and this guide sleeve is in the form of a cylinder of revolution portion and has an annular stop completing the contour of this sleeve, this annular stop being adapted to be able to abut axially against the stop of the bush to limit the axial penetration of the guide sleeve in the bush.

According to another characteristic of the invention, the base has a small plate designed to bear against a tooth of the patient adjacent to the area of the jawbone against which this base is placed.

The invention also concerns a method for production and a drilling assistance device characterized in combination by all or part of the characteristics mentioned above or below.

Other aims, characteristics and advantages of the invention will appear upon reading the following description, which refers to the appended figures in which:

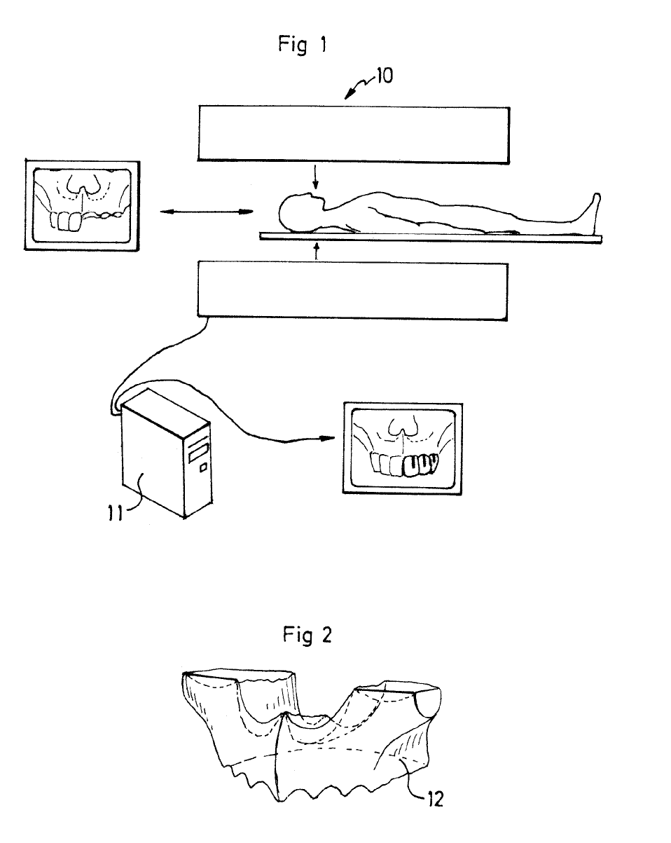

FIG. 1 is a diagram illustrating one step for obtaining tomographic data of a patient then used in a method for production of a drilling assistance device according to the invention,

FIG. 2 is a diagram illustrating one example of three-dimensional solid reproduction obtained in a method according to the invention,

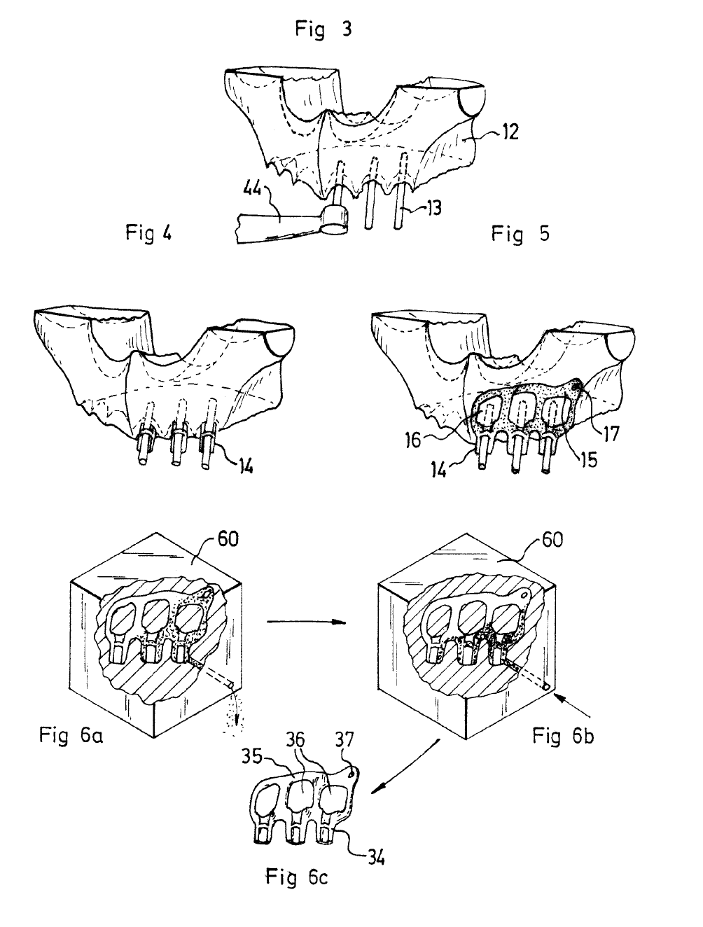

FIGS. 3 to 5 are diagrams illustrating the three successive steps, respectively, of producing a drilling assistance device model according to the invention from the three-dimensional solid reproduction of FIG. 2,

FIGS. 6a, 6b, 6c are diagrams illustrating three successive steps, respectively, of molding of a drilling assistance device according to the invention from the model obtained in FIG. 5,

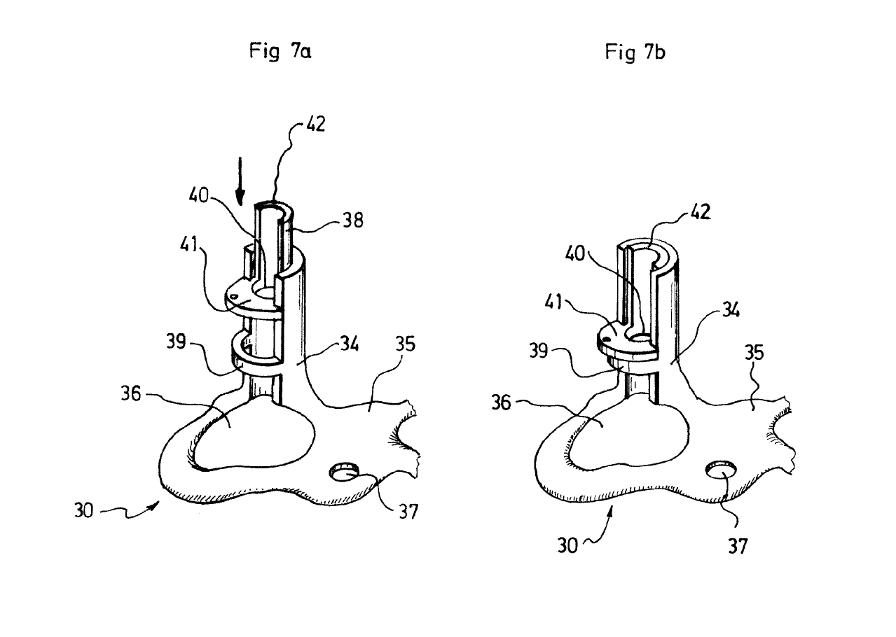

FIG. 7a is a perspective diagram illustrating a variation of embodiment of a drilling assistance device according to the invention, with a guide sleeve during placement, FIG. 7b illustrating this same device with the sleeve in place,

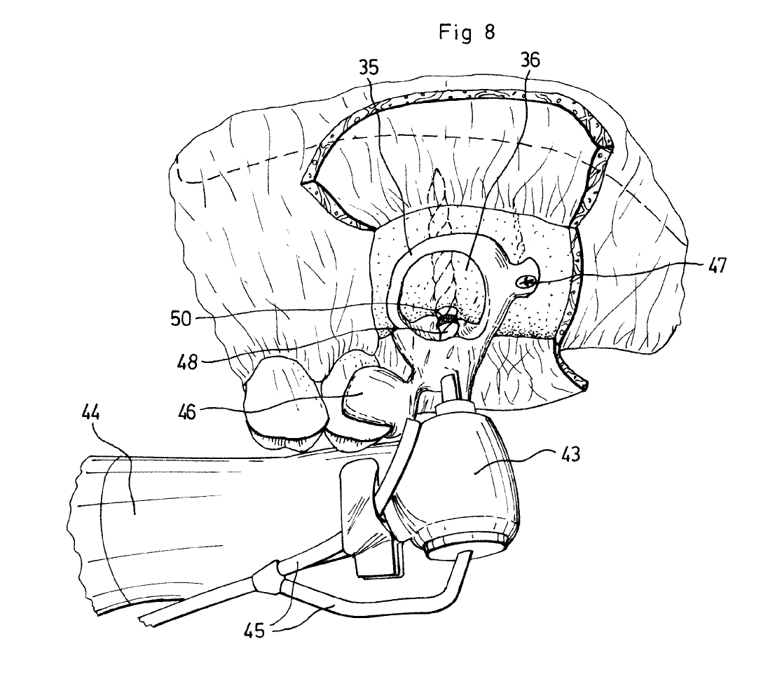

FIG. 8 is a perspective diagram illustrating another variation of embodiment of a drilling assistance device according to the invention during use for the drilling of an implant hole on a patient.

The method of the invention shown in FIGS. 1 to 6c makes it possible to produce a device 30 to assist with the positioning and realization by drilling of at least one implant hole, this device 30 having a rigid base 35 designed to come into contact with the bone structure, and for each implant hole, a drill guide 34, 38 comprising a bush 34 integral with the base and extending rigidly therefrom in one position, with an orientation and over a height precisely determined as described below.

FIG. 1 shows a step for obtaining tomographic data representative of an anatomical zone including a bone structure of a patient. To do this, one uses a piece of equipment 10 known in itself for tomodensitometry X (TDM) or computed tomography imaging. A piece of imaging equipment 10 of this type makes it possible to obtain digital data representative of transverse cross-sections of the anatomical zone, reconstructed from measuring the attenuation coefficient of the X-ray beam in the volume studied. This piece of equipment comprises a computer system 11 provided with auxiliary storage and microprocessor digital processing means, and different suitable software applications as described below.

Before performing the tomodensiometric examination of the patient, one takes an impression of the anatomical zone to undergo the implant, using a block of plastic material placed in the patient's mouth. From this impression, a plaster model is produced which makes it possible to study and define the shape and position of the implants. One then produces a provisional implant fitting. In the case of teeth, one makes a provisional dental fitting provided with radiopaque artificial teeth and comprising radiopaque median rods, for example in gutta percha, allowing visualization of their positions on the tomographic slices.

In one method according to the invention, one uses tomographic data representing slices of said anatomic zone with a distance between the slices less than or equal to 500 μm—in particular in the vicinity of 400 μm.

The images produced by the tomodensitometric appliance 10 are two-dimensional digital images measuring 512×512, or 262, 144 pixels. These images are framed, for a jaw, over an anatomical area having dimensions in the vicinity of 150 mm×150 mm=22,500 mm². Each pixel therefore represents, in this example, an anatomical area of approximately 300 μm on one side. The tomographic data therefore has a resolution in the vicinity of

$$\sqrt[3]{300 μ m \times 300 μ m \times 400 μ m} \approx 330 μ m .$$

The tomographic data is digital data in standard DICOM format. It is processed by a software application, for example VG STUDIO MAX® 1.2 32 bits marketed by the Company VOLUME GRAPHICS (HEIDELBERG, GERMANY). These data are images formed by pixels. The value of each pixel is proportional to the density of the material and therefore its nature. The software application VG STUDIO MAX® makes it possible to produce three-dimensional images of the anatomical area with the abovementioned resolution, as well as a meshing of this area done for this software application in standard stl format with a precision much greater than this resolution.

One then makes a three-dimensional solid representation 12 of the bone structure comprising at least the areas of this bone structure liable to receive an implant hole. This three-dimensional solid reproduction 12 can be made in the form of a sterolithographic reproduction through a method for rapid production of prototypes from files in stl format, using a 3D printer, for example reference EDEN® 350 marketed by the company OBJET GEOMETRIES (SINT-STEVENS-WOLUWE, BELGIUM). The precision of the surface state obtained for this three-dimensional solid reproduction 12 depends directly on the resolution of the digital meshing. Thus, with a digital meshing obtained as indicated above, one obtains a surface state with a precision less than or equal to 500 μm, in particular in the vicinity of 400 μm.

This three-dimensional solid reproduction 12 can be produced in any suitable material then making it possible to position the implant hole and shape the drilling assistance device as described below. In particular, the three-dimensional solid reproduction 12 is made in a material chosen in the group composed of polymeric synthetic materials, metallic alloys, plaster, ceramics, glues, cellulose, resins. Preferably, one uses a translucent or transparent material such as FULLCURE® 720 marketed by the company OBJET GEOMETRIES (SINT-STEVENS-WOLUWE, BELGIUM) making it possible to visualize the volume of this reproduction, the different peripheral surfaces and the passage of drills and tubes.

It should be noted that the three-dimensional solid reproduction 12 reproduces with great precision not only the surface state of the bone structure, but also the flaws, the density and the variations in density of the bone material within the volume of this bone structure as well as the anatomical obstacles (sinus cavities, nerves, foramen, . . . ). Thus, upon looking at this three-dimensional solid reproduction 12, the practitioner has a precise model of the bone structure on which he must then perform the implant. He can thus correctly choose the position, of each implant hole, the orientation of its axis, and, especially, its depth, while optimizing these different parameters to obtain a correctly positioned, functional implant with great mechanical aspect. For example, the practitioner performs, on the model, through a total drilling of the bone area where the implant must be placed from the bone ridge and up to the breach of the sinus or of the lower alveolar nerve. Then, he measures the exact distance between the breach point of the anatomical obstacles (alveolar sinus or canal) and the height of the bone ridge (by millimetric probe). He deduces from this measurement the necessary safety margin (approximately 2 mm), to obtain the maximum possible length of the implant hole. The drilling of the implant hole will be done at this exact depth.

The practitioner makes these different choices by drilling directly in the three-dimensional solid reproduction 12 as shown in FIG. 3. Preferably, these drillings are done with a no. 1 drill, i.e. a drill of minimum diameter.

The three-dimensional solid reproduction 12 thus provided with different drillings constitutes a faithful reproduction of the state of the bone structure which must be obtained during the surgical implant operation after performing different drillings with the first drill of minimum diameter.

After having done these drillings, a straight metallic rigid positioning rod 13 is introduced in each drill hole of the three-dimensional solid reproduction 12. Each positioning rod 13 is precisely adjusted to the diameter of each drilling, so as to be introduced into the latter substantially without play. The length of each positioning rod 13 is adapted so that, after having been entirely buried inside the drilling to the bottom thereof, this positioning rod 13 has a part which protrudes outside the three-dimensional solid reproduction 12 over a sufficient length, in particular greater than 3 mm, preferably in the vicinity of 1 cm to 2 cm. This part protruding to the outside has a diameter corresponding to that of the internal bore of each guide bush 34 having to be obtained, i.e. to the largest diameter of the drills able to be used during the surgical operation.

In the subsequent step shown in FIG. 4, a model 14 tubular bush is placed on each positioning rod 13. The internal diameter of this bush model 14 corresponds to the diameter of the external part of the positioning rod 13, the value of which corresponds to the diameter of the largest drill to be used. The length of each bush model 14 is greater than or equal to the maximum length which must be obtained for each bush 34 of the drilling assistance device 30 according to the invention then obtained from the model 14.

A base model 15 of the drilling assistance device is then shaped directly through modeling of a hardenable paste on the surface of the three-dimensional solid reproduction 12 around drillings and so as to extend each bush model 14, as shown in FIG. 5. This base model 15 can be produced through manual modeling by the practitioner, who can thus choose the appropriate surface portions on which the base 35 which will then be produced from this model 15, will be able to bear reliably, while allowing easy placement of this base on the bone structure, and, simultaneously, its later disassembly at the end of the operation. In particular, the practitioner can select the cortical parts of the bone surface to bear the maximum compression strain induced by the base 35. He can also visualize the assembly and disassembly problems, in particular the concave surface areas or those having undercut angles. He can also take into account the surgical constraints with which he will be faced, in particular the areas in which he will be able to make flaps to extricate the surface of the bone structure.

The base model 15 is adapted so that the base 35 is then connected to the base of each bush 34 while rigidly maintaining the latter in position and in its orientation. Moreover, the base model 15 comprises, across from each bush model 14, an opening 16 which reproduces an opening 36 arranged through the base 35 across from each drill hole, i.e. the base of each bush 34, each opening 36 thus formed then making it possible, during the surgical operation, to visualize the penetration of each drill 48 into the bone structure, the evacuation of drill dust and the passage and evacuation of an irrigation fluid outside the bone structure. In particular, each opening 36 is adapted to make it possible to visualize the penetration orifice 49 of the drill 48 into the bone structure as shown in FIG. 8. The drills 48 used generally being provided with drilling depth references, the practitioner can also control, during the surgical operation, the progression of the drill. As we see in the figures, despite the diagrammatic nature of these figures, the base 35 of the device 30 according to the invention is in fact present in the form of strips of peripheral seams defining the openings 36 whereof the dimensions are as large as possible to allow the passage of an outside irrigation fluid toward the bone surface and the passage of the drill dust toward the outside. The contact surface of these strips forming the base 35 on the bone structure must, however, be sufficient to ensure a sufficiently stable foundation of the device 30 on the bone structure, and, above all, to guarantee the fact that there is only one single possible placement location, given the precision of the reproduction of the surface state of this contact surface, which is closely combined with the surface state of the corresponding parts of the patient's bone structure.

In variation, nothing prevents one from providing that all or part of the bush 14 and/or base 15 models are made automatically or semi-automatically by a machine, for example with a digitally-controlled material spraying device according to a design developed digitally on a computer system using an appropriate software application.

The bush 14 and base 15 models are made in a modeling material which is plastic (in the case of manual modeling) but which is preferably hardenable, or in any case sufficiently rigid to then keep the modeled shape and enable its reproduction for the production of a model. This material is also chosen such that all of the specific surface irregularities of the three-dimensional solid reproduction 12, which correspond to those of the bone structure, are molded through the contact surface of the base model 15 on this three-dimensional solid reproduction 12. For example, one uses a material chosen from the group made up of waxes and silicones.

The base model 15 also has at least one piercing model 17 reproducing a piercing 37 then allowing the fixing of the base 35 on the bone structure using a screw 47 such as an osteosynthesis screw, or a rivet (for example in TEFLON®).

One thus obtains a model 14, 15 able to serve for the molding of an drilling assistance device 30 according to the invention. This model 14, 15 is detached from the three-dimensional solid reproduction 12, and can be the object of a molding method, for example of the lost wax type as shown in FIGS. 6a, 6b, 6c. In a variation not illustrated, one uses a production method through milling, or by injection molding, or by three-dimensional or other impression.

In the first step shown in FIG. 6a, one does an overmolding of the model 14, 15 by a refractory cement by arranging vents and/or conical jets traditionally, so as to obtain a mold 60 whereof the interior shapes correspond to those of the drilling assistance device 30 to be produced. After heating of this mold 60 at a suitable temperature, the model 14, 15 in heat-sensitive material (fusible or able to be burnt-on) disappears so as to allow the molding of the metallic alloy introduced in the melted state into the mold 60 as shown in FIG. 6b. After destruction of the mold 60, one obtains the reproduction in metallic alloy of the base 35 with each tubular bush 34 as shown in FIG. 6c.

The molding method making it possible to obtain the base 35 is chosen so as to be able to reproduce, with sufficient precision, all of the surface irregularities of the contact surface of the base model 15 on the solid reproduction 12, such that the contact surface of the base 35 on the bone structure then also has the combined surface irregularities of the latter. In particular, in a drilling assistance device 30 according to the invention, the surface state of the three-dimensional solid reproduction 12 reproduces the surface irregularities of the bone structure with a precision less than or equal to 500 μm—in particular in the vicinity of 400 μm, and the surface state of the contact surface of the rigid base 35 therefore has surface irregularities combined with those of the bone structure, this contact surface being produced with a precision less than or equal to 500 μm—in particular in the vicinity of 400 μm.

Each tubular bush 34 can then be severed at the desired length. One produces a solid model of the head 43 of the drilling tool (contra-angle) and of drill no. 1. This model is introduced into the tubular bush 34, which abuts on the head of the contra-angle. The length of the bush 34 is progressively reduced until the depth references of the drill in position in the solid reproduction 12 correspond to the required depth of the implant hole as determined as described above. Blockage of the head of the contra-angle on the upper part of the bush 34 makes excess drilling in depth impossible.

The structure thus produced of the assistance device 30 is taken for verification of all of these parameters on the solid reproduction 12. The model of the head of the contra-angle and of its drill must then perfectly penetrate the drill holes in axis and depth, guaranteeing good production of the device. One can then proceed with the intervention in total safety.

Each tubular bush 34 is adapted to be able to receive a tubular guide sleeve 38 of a drill 48. Each guide sleeve 38 is tubular in shape and adapted to be able to be inserted axially into the corresponding bush 34. Thus, the outer diameter of each guide sleeve 38 corresponds to the inner diameter of the bush 34, which is adapted to receive the drill of larger diameter.

In the embodiment shown in FIG. 7, the bush 34 is formed of a section of a half-cylinder of revolution (cut along a diametral axial plane) having an annular stop 39 completing the contour of this half-cylinder section, such that the axial end 40 of each sleeve 38 can be axially introduced into the bush 34 and through this annular stop 39. The annular stop 39 extends to a certain height relative to the base 35.

In the embodiment shown in FIGS. 7a and 7b, the annular stop 39 is made up of an annular portion. In the variation shown in FIG. 8, this annular stop 39 is made up of a radial surface of the axial end of a base portion of the bush 34 extending from the base 35.

Each guide sleeve 38 is also shaped in a section of a half-cylinder revolution (cut along a diametral axial plane) and also has an annular stop 41 completing the contour of this half-cylinder section, and adapted to abut axially against the stop 39 of the bush 34 to limit the axial penetration of the guide sleeve 38 into the bush 34. The axial length of each guide sleeve 38 is adapted so that the free end 42 of the latter the farthest from the base 35 serves as an axial stop in contact with which the head 43 of the drilling tool 44 can come at the end of drilling, so as to prohibit any excess drilling in depth. As shown in FIG. 7b, each guide sleeve 38 is the same height as the bush 34, the latter serving as a guide for the drill having the largest diameter.

The half-cylinder shape of the guide sleeve 38 and, if applicable, the bush 34, makes it possible to facilitate the insertion of the drill 48 in this guide sleeve 38 laterally. Each guide sleeve 38 is adapted to a particular drill diameter, i.e. has a cylindrical internal bore adapted to be able to receive and guide, axially without play, a drill 48. Thus, a drilling assistance device 30 according to the invention comprises a guide sleeve 38 for each drill diameter (with the exception of the drill having the largest diameter guided directly by the bush 34) and for each drilling to be done.

The drilling assistance device 30 thus obtained formed by the base 35, each bush 34, each guide sleeve 38 for each drill 48 to be used during the surgical operation can be precisely dimensioned and adjusted on the three-dimensional solid reproduction 12 on which it can be replaced, the practitioner being able to verify the proper position, proper orientation and proper maximum depth of drilling of each implant hole for each drill.

FIG. 8 shows an example of the use of a drilling assistance device 30 according to the invention. As one sees, the base 35 is fixed on the bone structure using an osteosynthesis screw 47. In the variation shown in FIG. 8, the base 35 also has a small plate 46 bearing on an adjacent tooth of the patient. Such a small plate 46 can also be made from the three-dimensional solid reproduction 12, the latter comprising a reproduction of the patient's teeth which can be done either simultaneously with the stereolithographic reproduction 12, or by artificial teeth added to this stereolithographic reproduction.

As one sees, the drilling tool 44 is equipped with internal (passing through the drill 48 axially) and external (projecting an irrigation fluid at the level of the orifice 49 for penetration of the drill 48 into the bone structure through the opening 36 of the base 35) irrigation tubes 45.

The base 35 bears directly on the bone structure, and not on the non-osseous tissues or the mucosa, and closely fits the surface state of this bone structure so as to be able to be placed on the bone structure in only one single possible location. As a result, during the surgical operation, placement of the drilling assistance device 30 is particularly simple and quick, no error being possible by the practitioner. During drilling, the practitioner can visualize, through the opening 36, the drill 48 penetrating into the bone structure. He can also visualize any references 50 formed on the drill 48 to check that the maximum drilling depth is not exceeded. Whatever the case may be, the device 30 according to the invention makes it possible to prevent drilling beyond a previously determined maximum depth. As a result, drilling can be done particularly quickly, the head 43 of the contra-angle abutting against the free end of the guide sleeve 38 or the bush 34, without particular precautions, in a single motion.

Each opening 36 also allows the evacuation of the drilling dust and the good circulation of irrigation fluids, preventing any untimely overheating liable to deteriorate the bone structure of the patient.

It goes without saying that the invention may be the object of a number of variations of embodiments relative to the examples shown in the figures and described above. In particular, while the invention is very advantageously applicable to the production of implant holes for the placement of dental implants, it can also be the object of application in other implant sites on humans or animals.

1. Method for producing a device, called drill assistance device, for assisting with the positioning and production by drilling of at least one implant hole in a bone structure of a patient for the placement of at least one implant, in which
one makes, from tomographic data representative of an anatomical zone, a three-dimensional solid reproduction (12) of said anatomical zone;
one makes, from the three-dimensional solid reproduction (12), a drill assistance device (30) comprising a rigid base (35) able to position itself and fix itself on the three-dimensional solid reproduction (12), this drill assistance device (30) comprising, for each drilling to be done, at least one drill guide (34, 38) able to define the position, the axial orientation and the depth of this drilling, each drill guide (34, 38) being rigidly supported by the base (35), characterized in that
the three-dimensional solid reproduction (12) consists of a reproduction of an internal and peripheral portion of said bone structure, exempt, at least in the vicinity of each implant hole, from the surrounding non-osseous tissues and mucosa,
the three-dimensional solid reproduction (12) is made with sufficient precision to reproduce the surface irregularities and the internal density variations of the bone structure,
said rigid base (35) having at least one surface, called contact surface, adapted to be able to bear in contact with an external surface portion of the three-dimensional solid reproduction (12), and therefore also later in contact with a peripheral surface portion of said bone structure,
said contact surface having a surface state adapted to fit the surface irregularities of this surface portion and so as to be able to fit onto this surface portion while being rigidly maintained in relation to said three-dimensional solid reproduction (12), and therefore also later on said bone structure, at least in part thanks to this fitting, in one single possible location.

2. Method according to claim 1, characterized in that said three-dimensional solid reproduction (12) is a stereolithographic reproduction.

3. Method according to claim 1, characterized in that said three-dimensional solid reproduction (12) is made in a translucent or transparent material making it possible to visualize the penetration of drills into this material.

4. Method according to claim 1, characterized in that one produces said three-dimensional solid reproduction (12) such that it has all of the areas of the bone structure liable to receive an implant hole.

5. Method according to claim 1, characterized in that said three-dimensional solid reproduction (12) is produced with a manufacturing uncertainty relative to the actual dimensions of the bone structure portion reproduced less than or equal to 500 μm—in particular in the vicinity of 400 μm.

6. Method according to claim 1, characterized in that, after having produced said three-dimensional solid reproduction (12), one produces a model (14, 15) of the drill assistance device (30) at least in part through modeling on the three-dimensional solid reproduction (12).

7. Method according to claim 6, characterized in that, after having produced said three-dimensional solid reproduction (12), one makes, for each implant hole, a drill hole in the three-dimensional solid reproduction (12), in one position, along an axis and with a depth corresponding to the position, the axis and the depth of the implant hole, respectively, then one places, in each drill hole, a positioning rod, then one produces a model (14, 15) of the drill assistance device (30) by placing a model (14) of each drill guide (34, 38) around each positioning rod and modeling a model (15) of the base around each drill guide model (14) thus placed, then one produces the drill assistance device (30) through molding from this model (14, 15).

8. Method according to claim 1, characterized in that each drill guide comprises a tubular bush (34) rigidly extending axially to extend the rigid base (35) while defining the position and the axial orientation of the drilling.

9. Method according to claim 8, characterized in that each bush (34) is adapted to be able to receive a tubular rigid sleeve (38) for guiding the drill, this rigid sleeve (38) being able to be inserted axially into the bush (34) until it abuts in a position where it is maintained rigidly without play relative to the bush (34), each guide sleeve (38) having a cylindrical internal bore with a diameter adapted to be able to receive and axially guide, without play, a drill.

10. Method according to claim 1, characterized in that each drill guide (34, 38) comprises a free stop end in contact with which a drill tool (43) may come, at the end of drilling, the axial length of the drill guide (34, 38) being adapted so as to prohibit any excess drilling in depth.

11. Method according to claim 1, characterized in that each drill guide (34, 38) comprises, as from a free stop end at least one axial portion having the shape of a half-cylinder of revolution portion, adapted to allow the lateral insertion of a drill in this drill guide (34, 38).

12. Method according to claim 1, characterized in that the base (35) has at least one opening (36) arranged across from the base of a drill guide (34, 38), adapted to allow the visualization of the penetration of a drill into the bone structure, the evacuation of drill dust and the passage of an irrigation fluid.

13. Method according to claim 1, characterized in that the base (35) has at least one perforation (37) for the passage of a fixing member (47) into the bone structure.

14. Method according to claim 1, characterized in that one produces the rigid base (35) such that it bears on the cortical parts of the bone structure allowing the assembly and disassembly of the rigid base (35) and across from the areas of maximum bone density.

15. Method according to claim 1, characterized in that the drill assistance device (30) is made of a metallic material.

16. Method according to claim 1, characterized in that one uses tomographic data representing slices of said anatomical area with a distance between the slices less than or equal to 500 μm—in particular in the vicinity of 400 μm.

17. Method according to claim 1, characterized in that, said bone structure being a maxillary structure, one produces a drill assistance device (30) adapted to allow the production of at least one dental implant hole.

18. Device, called drill assistance device (30), for assisting with the positioning and the production by drilling of at least one implant hole in a bone structure of a patient for the placement of at least one implant, comprising:
for each drilling to be done, at least one drill guide (34, 38) able to define the position, the axial orientation and the depth of this drilling,
a rigid base (35) having at least one surface, called contact surface, adapted to bear in contact with a peripheral surface portion of said bone structure, each drill guide (34, 38) being rigidly supported by the rigid base (35),
characterized in that said contact surface of the rigid base (35) has a surface state adapted to fit the surface irregularities of said peripheral surface portion of the bone structure and so as to be able to fit onto this surface portion while being rigidly maintained relative to said bone structure at least in part thanks to this fitting, in one single possible location.

19. Device according to claim 18, characterized in that the surface state of said contact surface of the rigid base (35) reproduces surface irregularities with a precision less than or equal to 500 μm—in particular in the vicinity of 400 μm.

20. Device according to claim 18, characterized in that each drill guide (34, 38) comprises a tubular bush (34) rigidly extending axially to extend the rigid base (35) while defining the position and the axial orientation of the drilling.

21. Device according to claim 20, characterized in that each bush (34) is adapted to be able to receive a tubular rigid sleeve (38) for guiding the drill, this rigid sleeve (38) being able to be inserted axially into the bush (34) until it abuts in a position where it is maintained rigidly without play relative to the bush (34), each guide sleeve (38) having a cylindrical internal bore with a diameter adapted to be able to receive and axially guide, without play, a drill.

22. Device according to claim 19, characterized in that each guide sleeve (38) has a free stop end in contact with which a drill tool (43) may come at the end of drilling, the axial length of the drill guide (34, 38) being adapted to prohibit any excess drilling in depth.

23. Device according to claim 18, characterized in that each drill guide (34, 38) has, as from its free stop end, at least one axial section of a half-cylinder of revolution adapted to allow the lateral insertion of a drill in this drill guide (34, 38).

24. Device according to claim 18, characterized in that the rigid base (35) has at least one opening (36) arranged across from the base of a drill guide (34, 38), adapted to allow the visualization of the penetration of each drill into the bone structure, the evacuation of drill dust and the passage of an irrigation fluid.

25. Device according to claim 18, characterized in that the rigid base (35) has at least one perforation (37) for the passage of a fixing member (47) into the bone structure.

26. Device according to claim 18, characterized in that the rigid base (35) is produced such that it bears on the cortical parts of the bone structure allowing the assembly and disassembly of the base rigid (35) and across from the areas of maximum bone density.

27. Device according to claim 18, characterized in that it is adapted to allow the production of at least one dental implant hole in a maxillary bone structure.

28. Device according to claim 18, characterized in that the base (35) has a reduced thickness, in the vicinity of 1 to 2 mm, relative to the height of each drill guide (34, 38); this height of each drill guide (34, 38) is in the vicinity of 5 to 15 mm.

29. Device according to claim 24, characterized in that at least one opening (36) is connected to the base of at least one drill guide (34, 38), with which it communicates.

30. Device according to claim 29, characterized in that said opening (36) is arranged so as to extend over at least one third of the area for connecting the drill guide (34, 38) to the rigid base (35).

31. Device according to claim 24, characterized in that at least one opening (36) is remote from the base of at least one drill guide (34, 38), and not communicate with this base.

32. Device according to claim 24, characterized in that at least one opening (36) has a length and a width, this length and this width being between three times and ten times the internal diameter of the drill guide (34, 38) across from the base of which this opening (36) is arranged.

33. Device according to claim 24, characterized in that the dimensions of at least one opening (36) are such that the opening (36) defines, with the edges of the rigid base (35), a peripheral border strip.

34. Device according to claim 24, characterized in that at least one drill guide (34, 38) is formed of a cylinder of revolution portion, defining a lateral opening, and in that an opening (36) arranged in the rigid base (35) extends in an extension of this lateral opening.

35. Device according to claim 18, characterized in that at least one drill guide (34, 38) is made up solely of one cylinder of revolution portion.

36. Device according to claim 35, characterized in that the drill guide (34, 38) comprises said bush (34) and in that this bush (34) is made up only of said cylinder of revolution portion and comprises an annular stop (39) completing the contour of this cylinder of revolution portion.

37. Device according to claim 36, characterized in that said annular stop (39) extends away from the area for connecting the bush (34) to the rigid base (35), in particular around one third of the height of this bush (34).

38. Device according to claim 36, characterized in that said annular stop (39) is made up of an annulus portion.

39. Device according to claim 36, characterized in that said annular stop (39) is made up of a radial surface of an axial end of one portion of the base of the bush (34), extending from the rigid base (35).

40. Device according to claim 36, characterized in that the drill guide (34, 38) comprises the aforementioned guide sleeve (38), and in that this guide sleeve (38) is in the form of a cylinder of revolution portion and has an annular stop (41) completing the contour of this sleeve (38), this annular stop (41) being adapted to be able to abut axially against the stop (39) of the bush (34) to limit the axial penetration of the guide sleeve (38) in the bush (34).

41. Device according to claim 18, characterized in that the rigid base (35) has a small plate (46) designed to bear against a tooth of the patient adjacent to the area of the jawbone against which this rigid base (35) is placed.
